(19) United States
(12) Patent Application Publication
(10) Pub. No.: US 2003/0120178 A1
(43) Pub. Date: Jun. 26, 2003

(54) Supplemental interlabial device

(75) Inventor: Yukio Heki, Kobe Hyogo (JP)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH

(21) Appl. No.: 10/302,167

(22) Filed: Nov. 22, 2002

Related U.S. Application Data
(60) Provisional application No. 60/342,696, filed on Dec. 20, 2001.

(51) Int. Cl. A61B 5/00

(52) U.S. Cl. 600/574

(57) ABSTRACT

The supplemental interlabial device to collect body fluids by disposing the device underneath or around an excretory orifice of a wearer is disclosed. The supplemental interlabial device has a longitudinal centerline, a transverse centerline, a wearer facing surface and an opposing surface. The device comprises a flange and an insertion protuberance. The flange is disposed opposite to the wearer's labia when the device is worn. The insertion protuberance is disposed inside the wearer's labia when the device is worn. The insertion protuberance is formed by a tube-like portion raised from the flange and has an opening at the top of the tube-like portion such that discharged body fluids pass through the opening.

### CROSS REFERENCE TO RELATED APPLICATION

This application claims the benefit of U.S. Provisional Application No. 60/342,696, filed on Dec. 20, 2001.

### FIELD OF THE INVENTION

The present invention relates to a supplemental device that is worn interlabially by female wearers for incontinence protection, catamenial purposes, or both. More particularly, the present invention relates to a supplemental device collecting discharged body fluids in cooperation with an absorbent article which is used to receive and absorb body fluids such as urine, menses, blood, and other fluids released from woman's body.

### BACKGROUND

Articles known for receiving and absorbing body fluid may be categorized into four types of construction. The first type of construction comprises an absorbent tampon which is inserted into the vagina. Body fluid is intercepted and absorbed by the inserted tampon so as not to reach the external wearer's skin around the vagina. An example of such internally-used tampon is disclosed in, e.g., U.S. Pat. No. 4,795,422 issued to Conner et al. However, in such a tampon, body fluid may leak past the tampon if heavy fluid flow occurs. Further, some wearers experience discomfort related to the use of a tampon, especially when the normal bulk of the tampon has been increased by absorption of body fluid.

The second type of construction comprises an externally-worn absorbent pad disposed on the outside of the body against the labia majora of the wearer. Such a pad intercepts and absorbs the body fluid at or about the introitus of the vagina, or outside the vagina after the fluid has left the vagina completely. An example of such an externally-worn absorbent pad is disclosed in, e.g., U.S. Pat. No. 3,888,254 issued to Hendricks. Externally-worn pads do not intrude into the highly sensitive introitus and the interior of the vagina. Thus, such pads may be more comfortable to the wearer when they are put in place first. However, when using such external pads, body fluid flows on the wearer's skin at and near the introitus before reaching the pad and being absorbed into the pad. The contact of body fluid with the external wearer's skin around the vagina causes discomfort. In addition, since the fluid is absorbed into the absorbent material in the pad through the body facing surface, the body facing surface is somewhat wet with body fluid. This is also uncomfortable to wearers. Thus, conventional external pads fail to prevent body fluid from contacting the wearer's skin around the vagina. In addition, the external pad is held in place by securing it to the user's undergarment. When the undergarment shifts, the pad also shifts. As a result, the pad is sometimes shifted out of its desired position around the introitus. This results in leakage of body fluid around the pad and onto the user's garments.

Accordingly, there has been considerable investigation into a third type of construction that can intercept body fluid both inside and outside of the vagina. Such articles comprise an internal tampon and an external pad in order to prevent the body fluid from reaching the wearer's skin around the vagina while addressing potential leakage onto the external wearer's skin around the vagina. An example of such a combination article is disclosed in, e.g., U.S. Pat. No. 3,905,372 issued to Denkinger. Such a combination article reduces leakage associated with the conventional tampons stated above because the article comprises an external pad to absorb body fluids besides the internal tampon. However, the internal tampon intruding into the introitus and the interior of the vagina cause discomfort to wearers. Furthermore, such an article does not address the issue of leakage around external pads.

As another example of the third type, U.S. Pat. No. 5,290,262 issued to Vukos et al. discloses a combination article comprising an external pad for absorbing body fluid and a nonabsorbent flexible sheath outwardly extending from the external pad. The sheath has height sufficient to be inserted into the vagina of a wearer when the external pad is placed against the wearer's labia majora. The nonabsorbent flexible sheath intercepts and diverts body fluid inside the wearer's body to direct body fluid downward into the external pad. Because the sheath is nonabsorbent, the article does not cause wearers discomfort of the conventional tampon which increases its bulk when it absorbs body fluids. However, the article does not address the issue of discomfort from an inserted foreign body into the vagina. In addition, because the discharged body fluid flows downward along the surface of sheath inserted into the vagina, the article disclosed in the Vukos et al. patent suffers from the drawback that the article fails to inhibit contact of body fluids with the inside of the labia which is highly sensitive to wetness of body fluid.

As an example of the fourth type of construction, U.S. Pat. No. 3,983,873 issued to Hirschman discloses a feminine hygienic pad for insertion into the interlabial space to absorb discharged body fluid. The pad is disposed into the interlabial space during use such that the entire device resides within the interlabial space. Because the pad does not intrude into the introitus and the interior of the vagina, the pad does not cause wearers discomfort from existence of an inserted foreign body into the vagina. However, the surface of the pad facing the body is somewhat wet with the discharged body fluid because the discharged body fluid is absorbed into the pad through the surface of the pad against the inside of the labia. In addition, in such a pad, body fluid may leak past the pad if heavy fluid flow occurs.

Thus, none of the publications above disclose an interlabial device that can solve the problem of discomfort from intrusion of a foreign body such as tampon into the vagina and discomfort from body fluid spreading onto the wearer's skin outside the vagina before being absorbed into the pad. In addition, none of the publications above disclose an interlabial device that can inhibit contact of body fluids with the inside of the labia efficiently. Thus, none of the existing arts provided all of the advantages and benefits of the present invention.

### SUMMARY

The supplemental interlabial device to collect body fluids by disposing the device underneath or around an excretory orifice of a wearer is disclosed. The supplemental interlabial device has a longitudinal centerline, a transverse centerline, a wearer facing surface and an opposing surface. The device comprises a flange and an insertion protuberance. The flange is disposed opposite to the wearer's labia when the device is worn. The insertion protuberance is disposed inside the wearer's labia when the device is worn. The insertion protuberance is formed by a tube-like portion raised from the flange and has an opening at the top of the tube-like portion such that discharged body fluids pass through the opening.

### BRIEF DESCRIPTION OF THE DRAWINGS

While the Specification concludes with claims which particularly point out and distinctly claim the invention, it is believed the present invention will be better understood from the following description of preferred embodiments taken in conjunction with the accompanying drawings, in which like reference numerals identify identical elements and wherein:

FIG. 1 is a perspective view of one embodiment of a supplemental interlabial device of the present invention;

FIG. 2 is a top view of the supplemental interlabial device of FIG. 1;

FIG. 3 is a cross-sectional view taken along line III-III of FIG. 2;

FIG. 4 is a cross-sectional view of another embodiment of a supplemental interlabial device of the present invention;

FIG. 5A is an exploded view of another embodiment of a supplemental interlabial device of the present invention;

FIG. 5B is a perspective view of the supplemental interlabial device of the embodiment of FIG. 5A;

FIG. 6 is a cross-sectional sagittal view of a human female wearer showing the placement of the interlabial device of the present invention in the wearer's interlabial space;

FIG. 7 is a transverse cross-sectional view of a human female wearer showing the placement of the interlabial device of the present invention in the wearer's interlabial device;

FIG. 8 is a perspective view of another embodiment of an interlabial device of the present invention;

FIG. 9 is a perspective view of one embodiment of a feminine hygiene product comprising the interlabial device of the present invention;

FIG. 10 is a schematic view of an exemplary embodiment of a system of an incontinence management system comprising the interlabial device of the present invention; and

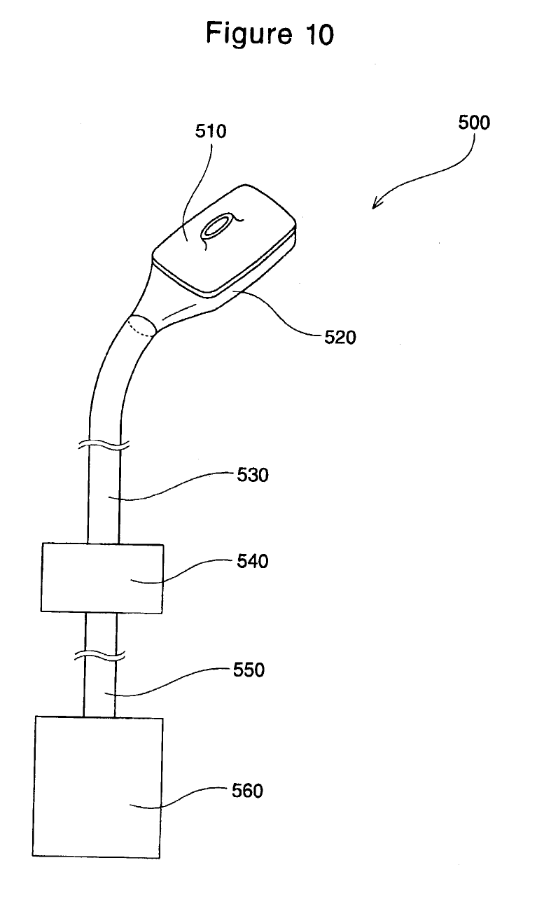

FIG. 11 is a schematic view of compression process for measuring rigidity of the interlabial device of the present invention.

### DETAILED DESCRIPTION

All cited references are incorporated herein by reference in their entireties. Citation of any reference is not an admission regarding any determination as to its availability as prior art to the claimed invention.

The definitions of several terms are first provided to assist the reader in understanding the present invention.

The term “comprising” means that other steps and other ingredients which do not affect the end result can be added. This term encompasses the term “consisting of” and “consisting essentially of”.

The term “disposable” as used herein, describes devices which generally are not intended to be laundered or otherwise restored or reused (i.e., they are intended to be discarded after a single use and, preferably, to be recycled, composted or otherwise disposed of in an environmentally compatible manner.)

The term “body fluid”, as used herein, includes all fluids released from a woman's body proximate the labia majora, including urine, menses, and blood.

The term “interlabial device”, as used herein, refers to a device which is specifically configured to reside within the interlabial space of female wearer during use. When the interlabial device is properly sized for an individual wearer, at least a part of the device of the present invention resides within such interlabial space.

The term “interlabial space”, as used herein, refers to that space in the pudendal region of the female anatomy which is located between the inside surface of the labia majora extending into the vestibule. Located within this interlabial space are the labia minora, the vestibule and the principal urogenital members including the clitoris, the orifice of the urethra, and the orifice of the vagina. Standard medical authorities teach that the vestibule refers to the space bounded laterally by the inside surfaces of the labia minora and extending interiorly to the floor between the clitoris and the orifice of the vagina. Therefore, it will be recognized that the interabial space as define above may refer to the space between the inside surfaces of the labia majora, including the space between the inside surfaces of the labia minora also known an the vestibule. The interlabial space for purposes of the present description does not extend substantially beyond the orifice of the vagina into the vaginal interior.

The term “labia”, as used herein, refers generally to both the labia majora and labia minora. The labia terminates anteriorly and posteriorly at the anterior commissure and the posterior commissure, respectively. It will be recognized by those skilled in the art that there is a wide range of variation among women with respect to the relative size and shape of labia mijora and labia minora. For purpose of the present description, however, such differences need not be specifically addressed. It will be recognized that the disposition of the interlabial device into the interlabial space of a wearer as defined above will require placement between the inside surfaces of the labia majora without regard to the precise location of the boundary between the labia majora and the labia mainora for a particular wearer. For a more detailed description of this portion of the female anatomy, attention is directed to Gray's Anatomy, Running Press 1901 Ed. (1974), at 1025-1027.

The term “excretory orifice”, as used herein, refers to an orifice which body fluids pass through to discharge the body fluids from the wearer's body when excretion occurs. Such an excretory orifice includes urethra, vaginal orifice, anus, and the like.

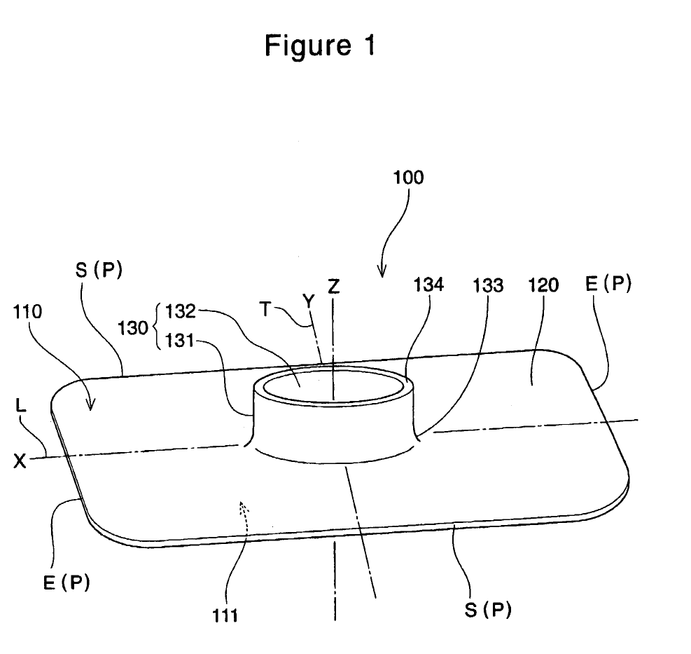

FIGS. 1 to 3 show one embodiment of a supplemental interlabial device of the present invention. The present invention, however, is not limited to a structure having the particular configuration shown in the drawings.

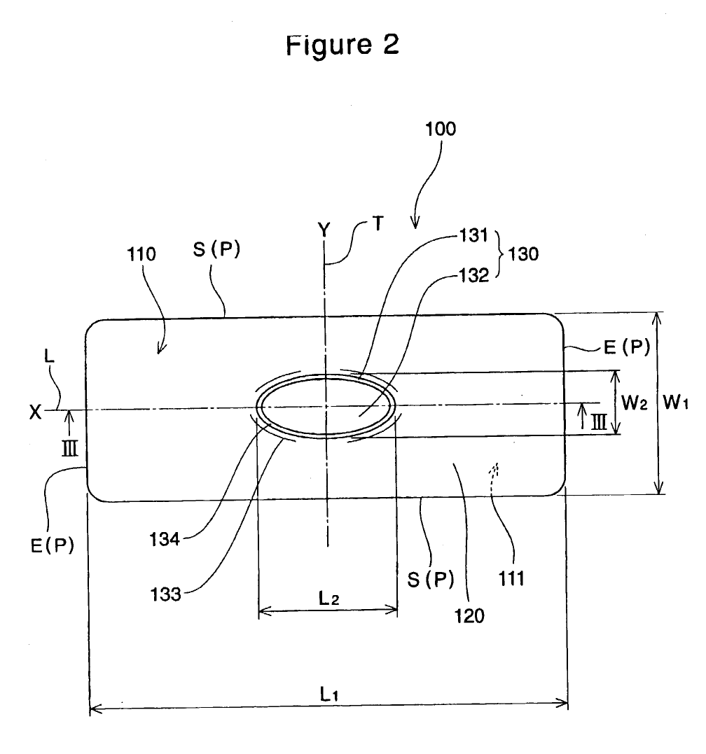

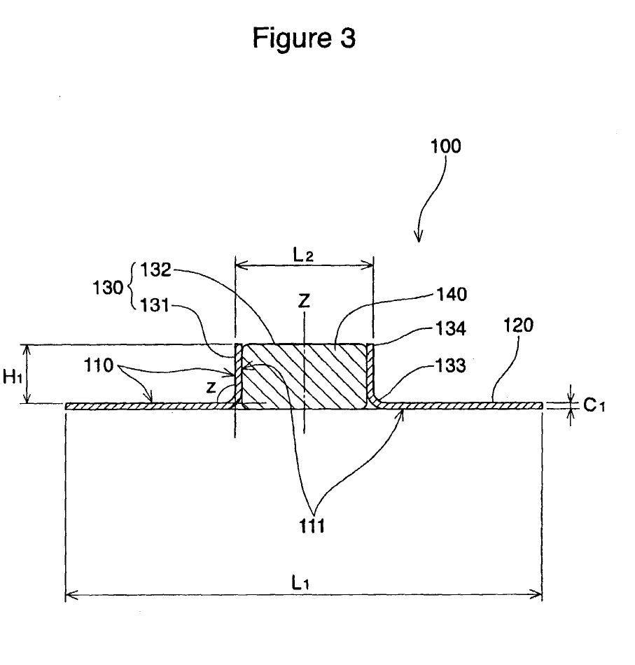

The supplemental interlabial device 100 shown in FIGS. 1 and 2 has a longitudinal centerline L which runs along the “X” axis. The term “longitudinal”, as used herein, refers to a line, axis or direction in the plane of the interlabial device 100 that is generally aligned with (e.g., approximately parallel to) a vertical plane which bisects a standing wearer into left and right body halves when the interlabial device 100 is worn. The terms “transverse”, “lateral”, or “Y direction” as used herein, are interchangeable, and refer to a line, axis or direction that is generally perpendicular to the longitudinal direction. The lateral direction is shown in FIG. 1 as the “Y” direction. The supplemental interlabial device 100 shown in FIG. 1 also has a transverse centerline T. The “Z” direction shown in FIGS. 1 and 3 is a direction parallel to the vertical plane described above. The term “upper” refers to an orientation in the Z-direction toward the wearer's head. The term “lower” refers to an orientation in the Z-direction toward the wearer's feet.

The supplemental interlabial device 100 shown in FIGS. 1 to 3 has two surfaces; one is a wearer facing surface 110 and the other is an opposing surface 111. The wearer facing surface 110 is the surface of the interlabial device 100 which is generally oriented toward the wearer when the interlabial device 100 is worn. The wearer facing surface 110 typically at least partially comes in contact with the wearer's skin during use of the interlabial device 100. The opposing surface 111 is the surface of the interlabial device 100 which is generally oriented away from the wearer when the interlabial device 100 is worn, and at least partially toward a garment if a garment is worn.

The supplemental interlabial device 100 comprises a flange 120, an insertion protuberance 130. The supplemental device 100 may further comprise a support member 140 as shown in FIG. 3. The flange 120 is disposed opposite to the wearer's labia when the device 100 is worn. The insertion protuberance 130 is disposed inside the wearer's labia when the device 100 is worn. The insertion protuberance 130 is formed by a tube-like portion 131 raised from the flange 120. The insertion protuberance 130 has an opening 132 at the top of the tube-like portion 131 such that discharged body fluids pass through the opening 132. The term “tube-like”, as used herein, refers to a hollow shape formed by a continuous side. Non limiting examples of cross sectional views of the hollow shapes formed by the continuous side include a circle, an oval, a semicircle, a square, a rectangle, a diamond, a sector, a triangle, a polygon, or any combination of the above. The support member 140 may be provided inside the insertion protuberance 130 to provide the insertion protuberance 130 with rigidity to maintain the opening 132 when the interlabial device 100 is worn. The interlabial device 100 may have a suitable size and shape that allows at least the majority of the insertion protuberance 130 to fit comfortably within the wearer's interlabial space and allows the flange 120 to cover around the outside of the interlabial space. When excretion occurs, the interlabial device 100 collects discharged body fluids such as urine, menses and other bodily exudates from the wearer's urethra, or vaginal orifice through the opening 132 and directs the discharged body fluids toward the outside of the interlabial space. Thus, the interlabial device 100 inhibits contact of the body fluids with the wearer's skin around the wearer's excretory orifice such as urethra or vaginal orifice during excretion. The interlabial device 100 described herein may be both flexible and compressible as long as the insertion protuberance 130 has rigidity to maintain the opening 132 when the interlabial device 100 is worn. Such flexibility and compressibility are important to comfort of the device 100. If the interlabial device 100 too flexible, the device 100 is not conveniently or easily placed in a desired position. If the interlabial device 100 is too stiff, it is uncomfortable for wearers to insert the device into the interlabial space.

The interlabial device 100 described above offers several advantages. The interlabial device 100 is particularly suited for reliable insertion by a variety of wearers. Since the interlabial device 100 does not intrude into the wearer's urethra and/or vagina, the device 100 does not cause wearers discomfort from existence of an inserted foreign body into the wearer's urethra and/or vagina. In addition, discharged body fluids are collected through the opening 132 of the interlabial device 100 within the interlabial space and flow toward the outside of the interlabial space through the inside of the device 100. Therefore, the interlabial device 100 can inhibit contact of body fluids with the inside of the labia which is highly sensitive to wetness of body fluid. Furthermore, the interlabial device 100 can work in cooperation with another absorbent article such as disposable underwears, disposable diapers (adult and baby) including pull-on diapers and training pants, disposable panties for menstrual use, and disposable absorbent pads including sanitary napkins. In such cases, discharged body fluids are separated from wearer's body by the interlabial device 100 and then absorbed/stored by another article capable of absorbing/storing the body fluids. Thus, the interlabial device 100 cooperating with another absorbent article can prevent rewet by discharged body fluids.

The size of the interlabial device 100 is important to its comfort and capability for collecting discharged body fluids. The length of the interlabial device 100 is measured along the longitudinal centerline L in the longitudinal direction (or “X”-direction). The interlabial device 100 may have an overall length of not less than about 5 mm, preferably not less than about 30 mm, more preferably not less than about 75 mm. The interlabial device 100 may also have an overall length of not more than about 300 mm, preferably not more than about 200 mm, more preferably not more than about 150 mm. The width of the interlabial device 100 is measured along the transverse centerline T in the transverse direction (or “Y”-direction). The interlabial device 100 may have an overall width of not less than about 9 mm, preferably not less than about 12 mm, more preferably not less than about 15 mm. The interlabial device 100 may also have an overall width of not more than about 200 mm, preferably not more than about 100 mm, more preferably not more than about 45 mm.

Construction of the interlabial device 100 according to the particular size parameters given above results in a product with increased comfort and effectiveness. For example, if the device is shorter (or smaller) than the particular size given above, it is difficult to position an interlabial device properly within the interlabial space. In addition, such a device may cause contact of body fluid with the external wearer's skin due to leakage of body fluid. In contrast, if a device is longer (or bigger) than the particular size given above, it is uncomfortable for wearers to insert the device into the interlabial space.

The flange 120 is formed in order to cover around the outside of the interlabial space when the interlabial device 100 is worn. The flange 120 has side edges S extending in the direction parallel to the Y axis and end edges E extending in the direction parallel to the X axis as shown in FIG. 1. The side edges S and the end edges E define the periphery P of the flange 120. The flange 120 does not intrude into the interlabial space when the interlabial device 100 is worn. Furthermore, the periphery P of the flange 120 is positioned outside the interlabial space to cover the labia minora, preferably the labia majora when the interlabial device 100 is worn. Thus, the flange 120 inhibits contact of discharged body fluids with the wearer's skin around the outside of the interlabial space. In addition, the flange 120 prevents the interlabial device 100 from intruding into the interlabial space too much in the Z direction. The flange 120 is preferably compliant, soft feeling, and non-irritating to the wearer's skin. The flange 120 should be liquid impermeable at least in a direction from the opposing surface 111 toward the wearer facing surface 110. The flange 120 may be also liquid impermeable completely. The flange 120 may have any suitable plan view configuration. Such suitable configurations include, but are not limited to: triangle shape; circle or oval shape; semicircle shape; sector shape; square, rectangular or diamond shape; pentagon shape or any combination of the above.

The flange 120 preferably has a length and width such that the flange 120 covers around the outside of the interlabial space sufficiently when the interlabial device 100 is worn. The flange 120 may have a length L1 of not less than to about 5 mm, preferably not less than about 30 mm, more preferably not less than about 75 mm. The flange 120 may also have a length L1 of not more than about 300 mm, preferably not more than about 200 mm, more preferably not more than about 150 mm. The flange 120 may have a width W1 of not less than to about 9 mm, preferably not less than about 12 mm, more preferably not less than about 15 mm. The flange 120 may also have a width W1 of not more than about 200 mm, preferably not more than about 100 mm, more preferably not more than about 45 mm. In addition, the flange 120 preferably has a thickness (or caliper) such that the interlabial device 100 does not cause discomfort to wearers when the interlabial device 100 is worn. The flange 120 may have a thickness C1 of not less than to about 0.1 mm, preferably not less than about 0.5 mm, more preferably not less than about 1.0 mm. The flange 120 may also have a thickness C1 of not more than about 7.0 mm, preferably not more than about 5.0 mm, more preferably not more than about 3.0 mm.

The insertion protuberance 130 is formed in order to collect discharged body fluids and to direct the discharged body fluids inside the interlabial device 100 when excretion occurs. The insertion protuberance 130 intrudes into the interlabial space when the interlabial device 100 is worn. The insertion protuberance 130 is formed by a tube-like portion 131 raised from the flange 120. The preferred shape of the tube-like portion 131 shown in FIGS. 1 to 3 (i.e., one in which the tube-like portion 131 is tapered at the ends) allows the insertion protuberance 130 to easily and comfortably fit the interlabial space. The tube-like portion 131 has a proximal end 133 joined to the flange 120 and a distal end 134 opposing to the proximal end 133. The term “joined” or “joining”, as used herein, encompasses configurations in which an element is directly secured to another element by affixing the element directly to the other element; configurations in which the element in indirectly secured to the other element by affixing the element to intermediate member(s) which in turn are affixed to the other element; and configurations in which one element is integral with another element, i.e. one element is essentially part of the other element. Therefore, the insertion protuberance 130 may be integral with the flange 120. Alternatively, the insertion protuberance 130 may be joined to the flange 120 as another element. The insertion protuberance 130 has an opening 132 at the top of the tube-like portion 131 such that discharged body fluids pass through the opening 132. Thus, the distal end 134 of the tube-like portion 131 surrounds the opening 132. The distal end 134 surrounding the opening 132 intrudes inside the interlabial space when the interlabial device 100 is worn. Therefore, discharged body fluids from the wearer's excretory orifice pass through the opening 132 within the interlabial space and are directed inside the interlabial device 100 without contact of the body fluids with the inside of the wearer's labia. The opening 132 may have any suitable plan view configuration. Such suitable configurations include, but are not limited to: triangle shape; circle or oval shape; semicircle shape; sector shape; diamond shape; pentagon shape or any combination of the above. The insertion protuberance 130 should be liquid impermeable at least in a direction from the opposing surface 111 toward the wearer facing surface 110. The insertion protuberance 130 may be also liquid impermeable completely.

The insertion protuberance 130 is preferably compliant, soft feeling, and non-irritating to the wearer's skin. In addition, the insertion protuberance 130 is preferably both flexible and compressible. Such flexibility and compressibility are important to comfort. If the insertion protuberance 130 is too stiff, the interlabial device 100 is uncomfortable to wearers. For example, when the wearer is in a sitting position, the insertion protuberance 130 may be forced forward against the wearer's clitoris and may cause discomfort to the wearer. If the insertion protuberance 130 is too flexible, it is difficult to maintain the opening 132 when the interlabial device 100 is worn. If the aperture of the opening 132 is not maintained (i.e. if the opening 132 is crushed), discharged body fluids can not be directed toward the inside of the device 100 through the opening 132. As a result, the body fluids may flow outside of the interlabial device 100 (i.e. between the wearer facing surface 110 and the wearer's skin). This may cause contact of the body fluids with the inside of the wearer's labia. Thus, the insertion protuberance 130 preferably has rigidity to maintain the aperture of the opening 132 when the interlabial device 100 is worn. The material of the insertion protuberance 130 may be selected to provide such rigidity. Alternatively, the insertion protuberance 130 may be provided with such rigidity by disposing the support member 140 inside the insertion protuberance 130.

The rigidity of the insertion protuberance 130 is measured by a compression tester. Thus, the term “rigidity”, as used herein, refers to the compression rate (%) measured by the following procedure (i.e. a ratio of a crushed thickness of a sample after compressional force is applied to an original thickness of a sample before compressional force is applied). The term “crushed thickness”, as used herein, refers to a value resulting from subtracting the thickness after transform from a thickness before transform. The compression tester is a device constructed in such a way that a gradually increasing load is smoothly applied to a sample until a predetermined force. The suitable instrument used for measurement is KES compression tester made by Kato-Tech in Japan. The following procedure is conducted under standard laboratory conditions at 23 degrees Centigrade (73 degrees Fahrenheit) and 50% relative humidity.

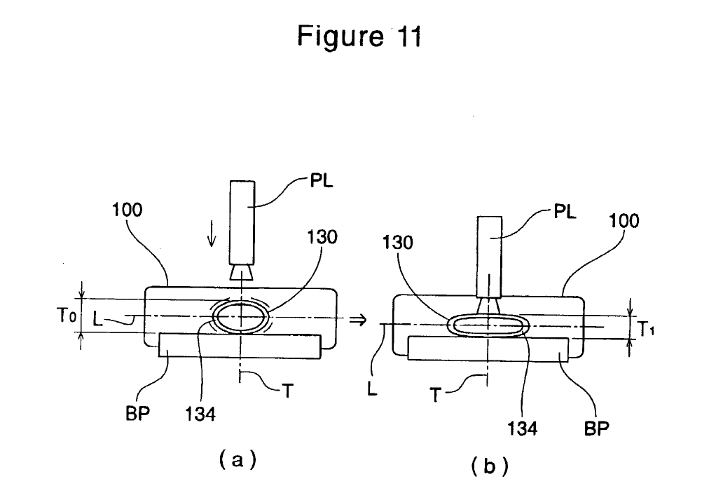

(1) Measure the original thickness (T0 mm) shown in FIG. 11(a) of the insertion protuberance 130 of the interlabial device 100. At least 3 samples should be prepared for the measurement.

(2) Set the speed (0.25 mm/min) and upper-limit force (100 gf/cm2).

(3) Put the insertion protuberance 130 of the interlabial device 100 on the bottom plate (BP) of the instrument as shown in FIG. 11(a).

(4) Turn on the start switch to lower plunger (PL) and compress the distal end 134 of the insertion protuberance 130 along the transverse centerline T at a constant rate as shown in FIG. 11(b).

(5) Read the thickness (T1 mm) shown in FIG. 11(b) of the insertion protuberance 130 of the interlabial device 100 when compressional force reaches the upper-limit force (100 gf/cm2).

(6) Repeat the above procedures (1) to (5) for the other samples.

(7) Calculate the average rigidity as follows.

Average Rigidity (%)=(T0−T1)×100/T0/3

The insertion protuberance 130 may have rigidity of not more than about 90(%), preferably not more than about 60(%), more preferably not more than about 30(%).

The insertion protuberance 130 preferably have a length and width such that the insertion protuberance 130 is comfortably and easily placed within the interlabial space when the interlabial device 100 is worn. The insertion protuberance 130 may have a maximum length L2 of not less than to about 5 mm, preferably not less than about 15 mm, more preferably not less than about 25 mm. The insertion protuberance 130 may also have a maximum length L2 of not more than about 100 mm, preferably not more than about 70 mm, more preferably not more than about 45 mm. The insertion protuberance 130 may have a maximum width W2 of not less than to about 3 mm, preferably not less than about 4 mm, more preferably not less than about 5 mm. The insertion protuberance 130 may also have a maximum width W2 of not more than about 30 mm, preferably not more than about 25 mm, more preferably not more than about 15 mm. In addition, the insertion protuberance 130 preferably have a height from the proximal end 133 to the distal end 134 such that the insertion protuberance 130 can sufficiently intrude into the interlabial space to collect discharged body fluids through the opening 132. The insertion protuberance 130 may have the height H1 from the proximal end 133 to the distal end 134 of not less than to about 5 mm, preferably not less than about 7 mm, more preferably not less than about 10 mm. In addition, the insertion protuberance 130 may also have the height H1 of not more than about 35 mm, preferably not more than about 25 mm, more preferably not more than about 15 mm.

The wearer facing surface 110 of the insertion protuberance 130 forms an angle Z with the wearer facing surface 110 of the flange 120 when the interlabial device 100 is worn. The angle Z may be from 30 to 150 degrees, preferably from 60 to 120 degrees, more preferably from 90 to 95 degrees. It is important that the angle Z is within the above-mentioned range in order to insert the insertion protuberance 130 into the interlabial space smoothly and to collect discharged body fluids through the opening 132 inside the interlabial space.

The interlabial device 100 is preferably compliant, soft feeling, and nonirritating to the wearer's skin, especially, the inside of the interlabial space. A suitable material of the interlabial device 100 may be manufactured from a wide range of materials such as woven and nonwoven materials; polymeric materials such as thermoplastic films, plastic films, and hydroformed thermoplastic films; foams such as polyurethane foams; reticulated foams; reticulated thermoplastic films; thermoplastic scrims; and impressive materials for dental. The interlabial device 100 is preferably made of a liquid impermeable material at least in a direction from the opposing surface 111 toward the wearer facing surface 110 in order to isolate discharged body fluids from the wearer's skin around the interlabial space during use of the device 100. In the embodiment shown in FIGS. 1-3, the interlabial device 100 comprises a single sheet formed by a nonwoven. Alternatively, the interlabial device 100 may comprise two or more sheets formed by nonwoven. Furthermore, the interlabial device 100 may be made of a single material formed by impressive materials for dental or polyurethane foams. Preferably, the nonwoven is made of liquid impermeable thermoplastic fibers in order to isolate discharged body fluids from the wearer's skin around the interlabial space during use of the device 100. Any plastic film, coating, or wax may be used, as long as it gives liquid impermeability to the interlabial device 100 at least in a direction from the opposing surface 111 toward the wearer facing surface 110.

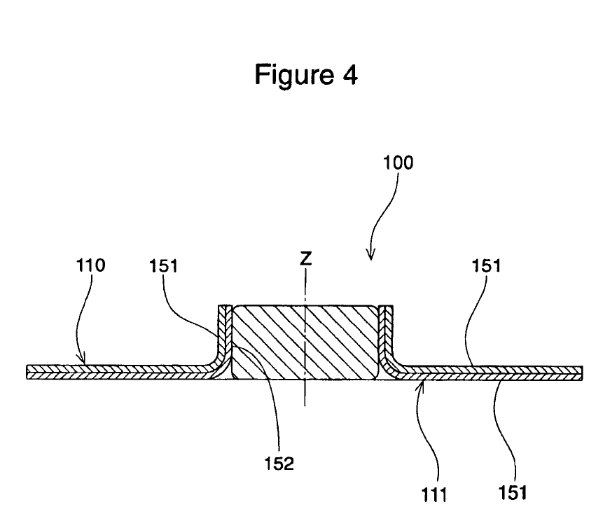

In the embodiment shown in FIGS. 1-3, the interlabial device 100 comprises a single layer. Alternatively, the interlabial device 100 may be formed by a plurality of layers, i.e., two or more layers. In one embodiment, the interlabial device 100 may comprise two layers comprising a wearer facing sheet 151 and an opposing sheet 152 as shown in FIG. 4. The wearer facing sheet 151 is oriented toward the wearer when the interlabial device 100 is worn. The wearer facing sheet 151 typically at least partially comes in contact with the wearer's skin during use of the interlabial device 100. The opposing sheet 152 is oriented away from the wearer when the interlabial device 100 is worn and at least partially toward a garment if a garment is worn. The material of the sheets 151, 152 may be formed with any material so that the interlabial device 100 is liquid impermeable. In particular, the sheets 151, 152 may comprise any material such as a nonwoven or a polymeric film. Preferably the wearer facing sheet 151 comprises a nonwoven and the opposing sheet 152 comprises a liquid impermeable polymeric film. The wearer facing sheet 151 comprising a nonwoven provides the wearer's skin inside the interlabial space with non-irritation and thus greatly improves the membrane healthiness in the interlabial space. The opposing sheet 152 comprising a polymeric film provides the interlabial device 100 with liquid impermeability. The interlabial device 100 may comprise three layers; one polymeric film layer and two nonwoven layers. In such an embodiment, the polymeric film may be interposed between the two nonwoven layers. This sequence of layers results in a closed fibrous structure, which has a particularly pleasing sensation on contact with the skin of the wearer.

Suitable woven and nonwoven materials for the material of the interlabial device can be comprised of natural fibers (e.g., wood or cotton fibers), synthetic fibers (e.g., polymeric fibers such as polyester, polypropylene, polyamide, polyolefin or polyethylene fibers) or from a combination of natural and/or synthetic fibers. The fibers may have any shape such as a circular cross section shape or a non-circular cross section shape, preferably a non-circular cross section shape. The fiber denier can be any of range depending up on the end use. Polyolefin polymer based nonwoven is beneficial in several reasons such as cost, processability into a form of fibers, or softness in the form of fibers.

The nonwoven web can be made by any known methods. It may be made by bonding of web-like arrays of fibers or filaments. The web may be made from fibers of discrete length ranging from few millimeter to few meters by carding or wet or air laying process or they may be produced by laying or blowing filaments as they are being melt extruded. The fabrics made by these latter process are commonly known as spunbonded or spunlaid and melt-blown nonwoven webs. A spunbonded nonwoven web may be defined generically as continuous filament fibrous structures which are made in the form of fabrics, sheets or tapes and are prepared from synthetic polymers in a process integrated with fiber manufacture. A melt-blown nonwoven web is a fibrous structure produced by extruding a polymer melt through a die into a high velocity stream of hot air to produce fine or super fine fibers which are deposited on a moving screen after quenching. A carded nonwoven web are made from webs of carded fibers. The preferred polymer for the production of filaments or fibers for making the nonwoven web is polyolefins such as polyethylene or polypropylene. In one embodiment, a preferred nonwoven web for the use of the material of the interlabial device of the present invention may be a spunbond-meltbrown-spunbond nonwoven comprising polypropylene fibers with a basis weight of between about 30 g/m2 and about 50 g/m2. An exemplary nonwoven web for the use of the present invention is supplied by Mitsui Chemical in Japan under the designation code of PQ1151 (commercial name: Syntex 15 gsm).

Suitable film materials for the material of the interlabial device may comprise a thermoplastic material. The thermoplastic material can be selected from among all types of polyolefins especially polyethylene, polypropylene, amorphous polyolefins, and the like; material containing meltable components comprising fibers or polymeric binders including natural fibers such as cellulose wood pulp, cotton, jute, hemp; synthetic fibers such as fiberglass, rayon, polyester, polyolefin, acrylic, polyamid, aramid, polytetrafluroethylene metal, polyimide; binders such as bicomponent high melt/low melt polymer, copolymer polyester, polyvinyl chloride, polyvinyl acetate/chloride copolymer, copolymer polyamide, materials comprising blends wherein some of the constituent materials are not meltable; air and vapour permeable materials including microporous films such as those supplied by EXXON Chemical Co., III, US under the designation EXXAIRE or those supplied by Mitsui Chemical Co., Japan under the designation ESPOIR NO; and monolithic breathable materials such as Hytrel™ available from DuPont and Pebax™ available from ELF Atochem, France. In a preferred embodiment, a film which is comprised in any layer is preferably permeable to gases such as air and to vapour such as water vapour in order to avoid the problem of entrapment and condensation of moisture vapour given off by the body of the wearer and thus, the hot, clammy and uncomfortable conditions after a short period of use.

The support member 140 may be disposed inside the insertion protuberance 130 in order to provide the insertion protuberance 130 with rigidity to maintain the opening 132. Alternatively, such a support member may be omitted from the interlabial device 100 if the insertion protuberance 130 can maintain the opening 132 without the support member 140 when the interlabial device 100 is worn. The support member 140 may have any suitable configuration as long as the insertion protuberance 130 can maintain the opening 132 when the interlabial device 100 is worn. Such suitable configurations include, but are not limited to: cylinder; sphere; cube; prism; diamond shape; pentagon shape or any combination of the above. Such a support member may be solid or hollow. In addition, the support member 140 may also have any suitable tube-like configurations. Preferably, the support member 140 rapidly collects discharged body fluids through the opening 132 and direct the body fluids toward outside the interlabial space. Since such body fluids are often discharged in gushes, the support member 140 has some gush handling capacity such that the support member 140 can rapidly receive practical quantities of discharged body fluids.

The gush handling capacity of the support member 140 is related to the void volume of the structure of the support member 140. The support member 140 should, therefore, be manufactured of a material that has sufficient void volume in the interstices or capillaries between the material or fibers to contain practical quantities of body fluid. Void volume within the support member 140 serves as a reservoir or “bucket” for large gushes of discharged body fluids with a minimum resistance to flow within the support member 140 such that the support member 140 can rapidly receive discharged body fluids.

It has been found that the support member 140 should have a percentage void volume greater than or equal to about 80%, preferably greater than or equal to about 90%, more preferably greater than or equal to about 95% (typically between about 93% and about 99%), such that there is sufficient void volume to contain in-use quantities of liquids or body fluids. The percentage void volume is calculated by the following equation:

Percentage void volume=(1−Vm/Vs)×100%

wherein Vm is the volume of the material determined by dividing the weight of the material or fibers in a given sample by the density of the material or fibers, and wherein Vs is the volume of the sample calculated by multiplying its area times its caliper measured under a load of 0.1 psi. Preferably, the support member 140 has a void volume of at least about 5 cm3, more preferably at least about 10 cm3, and most preferably at least about 15 cm3. In an especially preferred embodiment, the support member 140 has a void volume of about 20 cm3.

Various types of hydrophilic material can be used in the support member 140. Any type of hydrophilic fibers which are suitable for use in conventional absorbent products are also suitable for use in the support member 140. Examples of suitable materials of the support member 140 include comminuted wood pulp which is generally referred to as airfelt; creped cellulose wadding; meltblown polymers including coform; chemically stiffened, modified or cross-linked cellulosic fibers, synthetic fibers such as crimped polyester fibers; high surface area fibers; peat moss; tissue including tissue wraps and tissue laminates; foams such as polyurethane foams; sponges; or any equivalent material or combinations of materials, or mixtures of these. In one embodiment, the support member 140 may be formed by chemically stiffened, twisted, and curled bulking fibers; high surface area fibers; and binder fibers. An example of the support member 140 comprising these elements is described generally in U.S. Pat. No. 5,549,589 issued to Homey et al. on Aug. 27, 1996.

In a particularly preferred embodiment, the support member 140 comprises a nonwoven material comprising rayon fibers such as viscose rayon fibers and high surface area fibers such as eucalyptus fibers. Such high surface area fibers are useful to increase body fluids distribution in the support member 140. Such a nonwoven material preferably comprises between about 50% to about 70%, preferably about 65% staple length viscose rayon fibers, and between about 30% and about 50%, preferably about 35% of eucalyptus fibers. Suitable viscose rayon fibers are LYOCELL viscose rayon fibers, type 18453, obtained from Courtaulds Fibers, Inc. of North Axis, Ala. Suitable eucalyptus fibers are eucalyptus grandis, obtained from Aracruz, Brazil. The fibers may be blended together and formed into a web by a variety of methods including wet-laying method, air-laying method, carding, needle punching or other methods. Air-laying method and needle punching are particularly preferable.

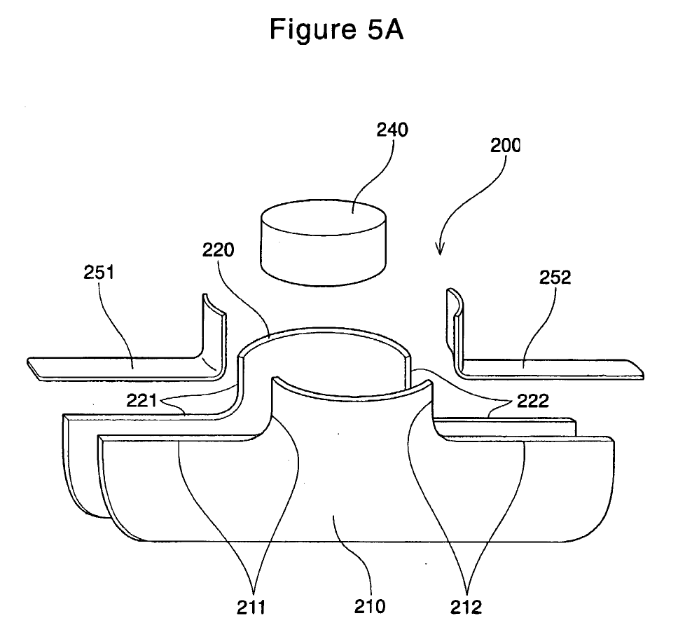

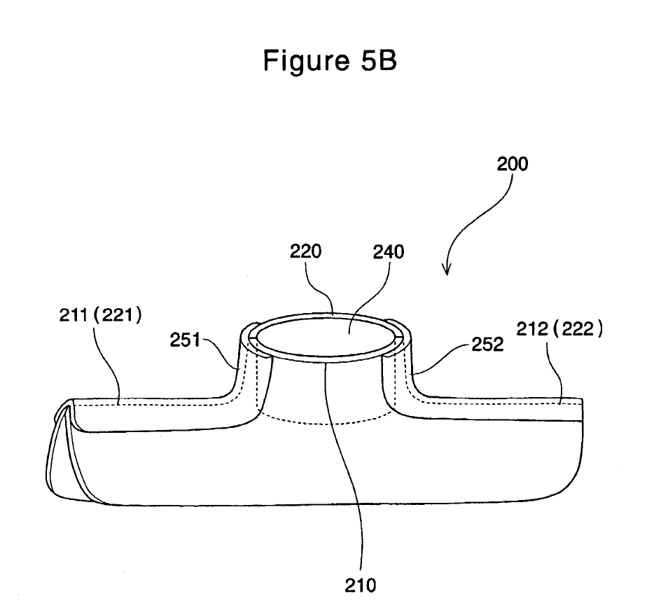

The interlabial device of the present invention may comprise several components as shown in FIGS. 5A and 5B. In the embodiment shown in FIGS. 5A and 5B, the interlabial device 200 comprises a first sheet 210 and a second sheet 220. In addition, the interlabial device 200 may further comprise a support member 240. The first and second sheets 210 and 220 are bonded together by any means known in art, such as a heat seal or glue in order to form the interlabial device 200. A suitable material of the first and second sheets 210 and 220 may be manufactured from any above-mentioned materials that are applicable to the interlabial device shown in FIGS. 1 to 4. In the embodiment shown in FIGS. 5A and 5B, the first and second sheets 210 and 220 comprise a single layer respectively. Alternatively, the first and second sheets 210 and 220 may be formed by a plurality of layers, i.e., two or more layers. In a preferred embodiment, the first and second sheets 210 and 220 may comprise two layers comprising a nonwoven and a liquid impermeable polymeric film. The first and second sheets 210 and 220 are preferably bonded together along edges 211, 212, 221 and 222 of the first and second sheets 210 and 220 by any means known in the art such as a heat seal or glue. The interlabial device 200 may further comprise a first seal member 251 and a second seal member 252. The first and second seal members 251 and 252 cover the edges 211, 212, 221 and 222 to provide the interlabial device 200 with liquid impermeability at the edges 211, 212, 221 and 222 as shown in FIG. 5B. The materials of the first and second seal members 251 and 252 may be manufactured from any materials as long as they promote liquid impermeability of the interlabial device 200 at the edges 211, 212, 221 and 222. The material of the seal members 251 and 252 may be identical with the material of the first and second sheets 210 and 220. Alternatively, the material of the seal members 251 and 252 may be different from the material of the first and second sheets 210 and 220.

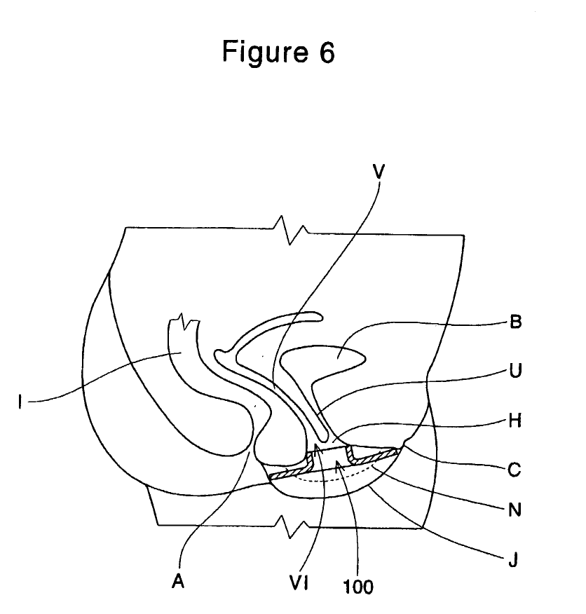

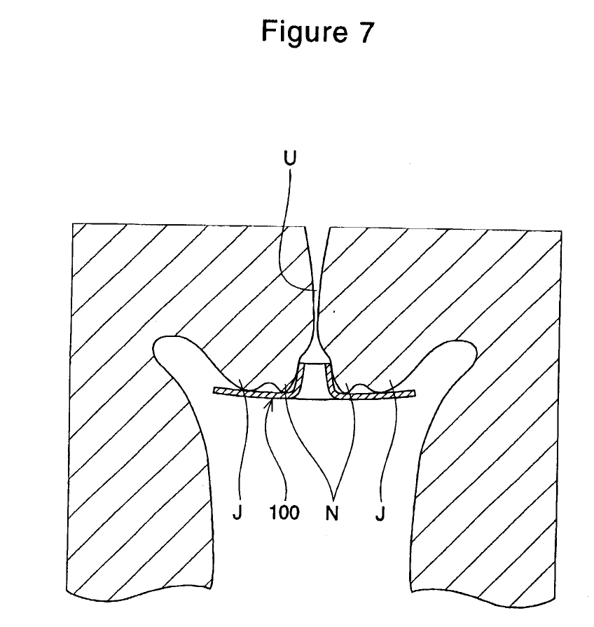

FIGS. 6 and 7 show a preferred embodiment of the interlabial device 100 of the present invention inserted into the interlabial space of a wearer. The urogenital members shown in FIGS. 6 and 7 include the bladder B, the vagina V, the urethra U, the clitoris C, the large intestine 1, the anus A, the vagina introitus VI, the hymeneal ring H, the labia minora N, and the labia majora J. FIGS. 6 and 7 show the relationship of these anatomical features of the wearer to the intelabial device 100 when the device 100 is properly inserted for use. Once the interlabial device 100 is inserted, the wearer facing surface 110 of the interlabial device 100 contacts the inside surfaces of the labia. Therefore, discharged body fluids tend to pass through the opening 132 rather than flowing between the wearer facing surface 110 of the device 100 and the inside surfaces of the labia. As a result, the interlabial device 100 inhibits contact body fluids with the wearer's skin inside the interlabial space. In addition, since the interlabial device 100 does not intrude into the wearer's urethra and/or vagina as shown in FIGS. 6 and 7, the device 100 does not cause wearers discomfort from existence of an inserted foreign body into the wearer's urethra and/or vagina. The flange 120 of the interlabial device 100 is positioned outside the interlabial space so as to cover the labia minora, preferably the labia majora when the interlabial device 100 is worn. Thus, the interlabial device 100 can inhibit contact of body fluids with the wearer's skin around the outside of the interlabial space. The interlabial device 100 is preferably at least partially retained in place by exerting a laterally outwardly oriented pressure on the inner surfaces of the wearer's labia minora, labia majora, or both. Additionally, the interlabial device 100 may also be held by attraction of naturally moist labial surfaces to the wearer facing surface 110 of the device 100. Optionally, the wearer facing surface of the interlabial device 100 may be provided with a biocompatible adhesive in order to assist the adhesion of the device 100 to inside surfaces of the wearer's labia. The strength of such an adhesive should be selected such that it is possible to remove the device 100 from the wearer's interlabial space easily and comfortably.

The interlabial device of the present invention may be employed in cooperation with another absorbent article such as disposable underwears, disposable diapers (adult and baby) including pull-on diapers and training pants, disposable panties for menstrual use, and disposable absorbent pads including sanitary napkins. Such an absorbent article may be any suitable conventional absorbent article. In such cases, discharged body fluids are separated from wearer's body by the interlabial device and then absorbed/stored by another article capable of absorbing/storing the body fluids. Thus, the interlabial device used with absorbent articles prevents rewet by discharged body fluids. Additionally, superior performance in reducing body and clothing soiling over extended periods of wear time such as overnight can be obtained by using the interlabial device used with absorbent articles. The interlabial device and absorbent articles may be packaged in a common package as a feminine hygiene “kit”. Such a kit facilitates use of the interlabial device cooperating with absorbent articles. In order to use an interlabial device cooperating with absorbent articles (e.g. a sanitary napkin), the wearer inserts the interlabial device into her interlabial space and places the sanitary napkin in the crotch portion of a panty-type undergarment. These two steps may be performed in either order. Some women will prefer to place the sanitary napkin in the panty crotch first in order to catch and absorb and drops of body fluids flow which might be released prior to the time that the interlabial device is inserted. Other women will chose to first insert the interlabial, device. After the interlabial device is inserted and the sanitary napkin positioned in the undergarment crotch, the undergarment is pulled up into its usual wearing position. Consequently, the sanitary napkin will be worn simultaneously with the interlabial device.

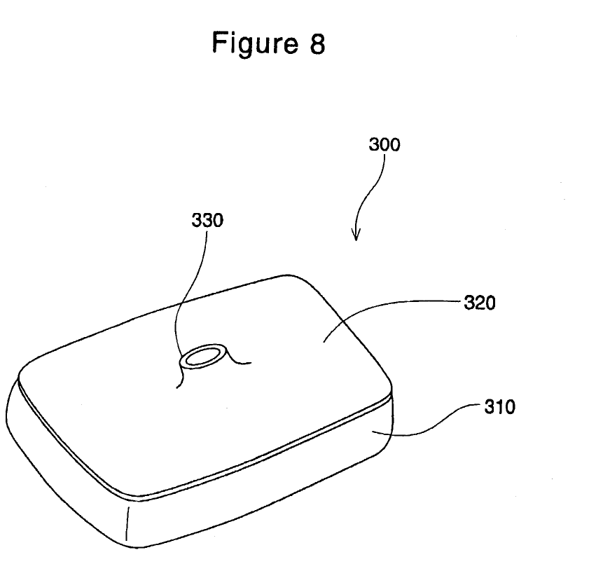

In another preferred embodiment, the interlabial device of the present invention may further comprise a liquid impermeable backsheet. As shown in FIG. 8, the interlabial device 300 comprises a liquid impermeable backsheet 310, a flange 320 and an insertion protuberance 330. The liquid impermeable backsheet 310 is joined to the flange 320 by any means known in the art such as a heat seal or glue. Thus, the liquid impermeable backsheet 310 functions as a pouch to store discharged body fluids in cooperation with the flange 320. The material of the liquid impermeable backsheet 310 may be identical with that of the flange 320 and/or the insertion protuberance 330. Alternatively, the material of the liquid impermeable backsheet 310 may be different from that of the flange 320 and/or the insertion protuberance 330. In the embodiment, the interlabial device 300 can be worn as a “stand alone” product because the device 300 is capable of storing discharged body fluids.

An absorbent material may be further contained within the device 300. The absorbent material may be positioned inside the backsheet 310 in any suitable manner. For example, the absorbent material may be loosely arranged within the device 300 or may be secured to the inner side of the backsheet 310. Any known techniques for securing absorbent material to nonwoven and film substrates may be used to secure the absorbent material to the inner side of the backsheet 310. The absorbent material may also be arranged to have any desired shape or configuration (e.g., rectangular, oval, circular, etc.). The absorbent material may comprise any absorbent material which is capable of absorbing and retaining discharged body fluids. The absorbent material may comprise a wide variety of liquid-absorbent materials commonly used in disposable diapers and other absorbent articles such as comminuted wood pulp, which is generally referred to as airfelt. Examples of other suitable absorbent materials include creped cellulose wadding; meltblown polymers, including coform; chemically stiffened, modified or cross-linked cellulosic fibers, synthetic fibers such as crimped polyester fibers; peat moss; tissue, including tissue wraps and tissue laminates; absorbent foams; absorbent sponges; superabsorbent polymers; superabsorbent hydrogel-forming polymeric material; absorbent gelling materials; or any other known absorbent material or combinations of materials or mixtures of these. The configuration and construction of the absorbent component may also be varied (e.g., the absorbent component may have varying caliper zones (e.g., profiled so as to be thicker in the center), hydrophilic gradients, superabsorbent gradients, or may comprise one or more layers or structures.

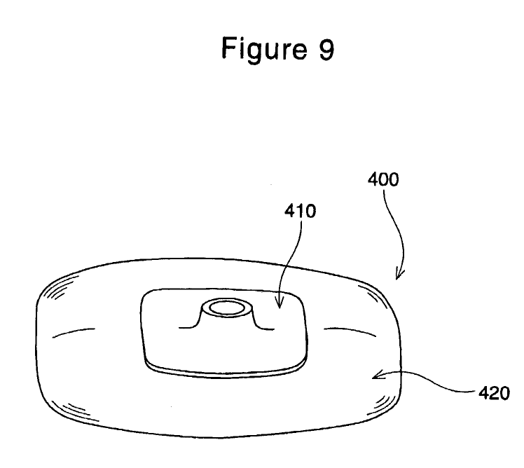

The interlabial device of the present invention may also be used as part of feminine hygiene products. In one preferred embodiment, the feminine hygiene product 400 comprises an interlabial device 410 to collect discharged body fluids and a pouch 420 to store discharged body fluids as shown in FIG. 9. In the embodiment, the interlabial device 410 is joined to the pouch 420 by any means known in the art such as a heat seal or glue. The material of the pouch 420 may be manufactured from any materials as long as the pouch 420 can store discharged body fluids therein. The material of the pouch 420 may be identical with that of the interlabial device 410. Alternatively, the material of the pouch 420 may be different from that of the interlabial device 410.

The pouch 420 is a flexible receptacle for the containment of discharged body fluids. The pouch 420 can be provided in any shape or size depending on the intended use thereof, i.e. whether the product 400 is intended for bedridden patients or active patients suffering from incontinence. For example an elongated pouch which is principally tubular or rectangular is typically utilized by bedridden patients and elderly incontinence sufferers. For more active wearers whether infants or adults, the pouch 420 of the product 400 should preferably be anatomically shaped such that the pouch 420 follows the contours of the wearer's body and can be worn inconspicuously by the wearer under normal garments. The pouch 420 may have any suitable configuration as long as the pouch 420 can store discharged body fluids. Such suitable configurations include, but are not limited to: cylinder; sphere; cube; prism; diamond shape; pentagon shape or any combination of the above.

The pouch 420 is preferably designed to provide sufficient volume for discharged body fluids under a variety of wearing conditions, also when worn by a freely moving, i.e., not bedridden wearer. The pouch 420 is designed to safely contain any entrapped body fluids, typically it will be liquid impermeable, yet it may be breathable. The pouch 420 is designed of sufficient strength to resist rupturing in use. Depending on the shape of the pouch 420 required, the pouch may be made from a unitary piece of material or from a number of separate pieces of material, which may be identical or different and which are sealed at their respective peripheries.

The pouch 420 may comprise one or multiple layers, preferably two or three layers. The layer on the inside of the bag, which will typically at least partially come in contact with discharged body fluids, is called the inner layer. The outermost layer of the pouch 420, which will typically at least partially come in contact with the skin of the wearer and the garments of the wearer, is called the outer layer. The layer of the pouch material may be provided from any material so that the pouch 420 is liquid impervious. The layer may in particular comprise any material such as a nonwoven or a polymeric film. In a preferred embodiment, the layer may be formed from a laminate comprising a nonwoven layer and a polymeric film. The outer layer of the pouch 420 is preferably provided with a nonwoven layer. The nonwoven outer layer does not provide the wearer's skin with any irritation and thus significantly reduces the problem of occlusion and greatly improves skin healthiness. In one preferred embodiment, the pouch 420 comprises two layers. Preferably the outer layer comprises a nonwoven layer and the inner layer comprises a liquid impermeable film. Alternatively, the pouch 420 comprises three layers; one film layer and two nonwoven layers. The film may be interposed between the two nonwoven layers. This sequence of layers results in a closed fibrous structure, which has a particularly pleasing sensation on contact with the skin of the wearer.

The absorbent material stated above may be further contained within the pouch 420. The absorbent material may be positioned inside the pouch 420 in any suitable manner. For example, the absorbent material may be loosely arranged within the pouch 420 or may be secured to the inner side of the pouch 420. Any known techniques for securing absorbent material to nonwoven and film substrates may be used to secure the absorbent material to the inner side of the pouch 420.

The interlabial device of the present invention can be employed as part of an incontinence management system. The incontinence management system 500 comprises an interlabial device 510, a pouch 520, a first pipe 530, a pump 540, a second pipe 550 and a storage bag 560. The interlabial device 510 joined to the pouch 520 collects discharged body fluids and directs the body fluids toward the inside of the pouch 520. The pump 540 sucks the body fluids stored into the pouch 520 through the first pipe 530, and then directs the body fluids toward the storage bag 560 through the second pipe 550. The storage bag 560 receives the body fluids from the pump 540 and stores the body fluids therein. The incontinence management system 500 can be employed for bedridden patients suffering from incontinence. Alternatively, the system 500 may be also employed for active patients suffering from incontinence as a “wearable” toilet. Preferably, all the components of the system 500 (i.e. the interlabial device 510, the pouch 520 and the first pipe 530, the pump 540, the second pipe 550 and the storage bag 560) may easily be attached to the system 500 or removed from the system 500 respectively for disposable use. By using the interlabial device of the present invention as part of an incontinence management system, it is possible to obtain superior performance in reducing body and clothing soiling over extended periods of wear time.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A supplemental interlabial device to collect body fluids by disposing the device underneath or around an excretory orifice of a wearer, the device having a longitudinal centerline, a transverse centerline, a wearer facing surface and an opposing surface, the device comprising a flange and an insertion protuberance,
the flange disposed opposite to the wearer's labia when the device is worn,
the insertion protuberance disposed inside the wearer's labia when the device is worn, the insertion protuberance formed by a tube-like portion raised from the flange and having an opening at the top of the tube-like portion such that discharged body fluids pass through the opening.

2. A supplemental interlabial device of claim 1 wherein the flange has side edges extending in a transverse direction and end edges extending in a longitudinal direction.

3. A supplemental interlabial device of claim 1 wherein the device is made of a material which is liquid impermeable at least in a direction from the opposing surface toward the wearer facing surface.

4. A supplemental interlabial device of claim 1 wherein the insertion protuberance has rigidity to maintain the opening when the device is worn.

5. A supplemental interlabial device of claim 4 wherein the insertion protuberance has the rigidity which is not more than 30(%).

6. A supplemental interlabial device of claim 5 wherein the insertion protuberance has the rigidity which is not more than 60(%).

7. A supplemental interlabial device of claim 1 wherein the device comprises a support member disposed inside the insertion protuberance to maintain the opening when the device is worn.

8. A supplemental interlabial device of claim 7 wherein the support member has a function acquiring discharged body fluids through the opening.

9. A supplemental interlabial device of claim 8 wherein the support member is made of polyurethane foam.
